(19) United States
(12) Patent Application Publication
(10) Pub. No.: US 2025/0064582 A1
(43) Pub. Date: Feb. 27, 2025

(54) LEAFLET THINNING METHOD FOR ACCURATE FRAME ATTACHMENT

(71) Applicant: Boston Scientific Scimed, Inc., Maple Grove, MN (US)

(72) Inventors: Michael G. Hayes, Salthill (IE); Joseph Murphy, Co Galway (IE); Declan Loughnane, Galway (IE); Tim O'Connor, Galway (IE)

(21) Appl. No.: 18/810,141

(22) Filed: Aug. 20, 2024

Related U.S. Application Data
(60) Provisional application No. 63/533,777, filed on Aug. 21, 2023.

(51) Int. Cl. A61F 2/24

(52) U.S. Cl. CPC A61F 2/2415; A61F 2/2418; A61F 2240/001

(57) ABSTRACT

Embodiments herein relate to methods of attaching leaflets to a valve frame and valves made using the same. In an embodiment, a method of making an implantable prosthetic valve is included. The method can include compressing at least a portion of valve leaflet tabs, inserting the compressed valve leaflet tabs into commissural slots of a valve frame, and expanding the compressed valve leaflet tabs. Other embodiments are also included herein.

This application claims the benefit of U.S. Provisional Application No. 63/533,777 filed Aug. 21, 2023, the content of which is herein incorporated by reference in its entirety.

### TECHNICAL FIELD

Embodiments herein relate to methods of attaching leaflets to a valve frame and valves made using the same.

### BACKGROUND OF THE INVENTION

The human body has four heart valves: a pulmonary valve, a tricuspid valve, an aortic valve and a mitral valve. Heart valves function to allow blood to flow in a particular direction from one chamber to another through the heart and from the heart into the major blood vessels connected to the heart, such as the aorta and pulmonary artery. As the heart muscle contracts and relaxes, the valves open and shut, letting blood flow into the ventricles and atria at alternate times.

Heart function can be significantly impaired when a heart valve is not working properly. Heart valve disease can be congenital, but can also occur in adults due to various causes. Heart valve issues can include regurgitation (blood leaking backwards), stenosis (narrowed valve openings), and atresia. Fortunately, prosthetic heart valves can be implanted to address heart valve issues and restore proper heart function.

### SUMMARY OF THE INVENTION

Embodiments herein relate to methods of attaching leaflets to a valve frame and valves made using the same. In a first aspect, a method of making an implantable prosthetic valve is included. The method can include compressing at least a portion of valve leaflet tabs, inserting the compressed valve leaflet tabs into commissural slots of a valve frame, and expanding the compressed valve leaflet tabs.

In a second aspect, in addition to one or more of the preceding or following aspects, or in the alternative to some aspects, compressing at least a portion of valve leaflet tabs includes removing liquid from the valve leaflet tabs.

In a third aspect, in addition to one or more of the preceding or following aspects, or in the alternative to some aspects, removing the liquid from the valve leaflet tabs includes removing at least 50% by weight of the starting liquid content.

In a fourth aspect, in addition to one or more of the preceding or following aspects, or in the alternative to some aspects, the liquid can include an aqueous solution.

In a fifth aspect, in addition to one or more of the preceding or following aspects, or in the alternative to some aspects, the liquid can include a glutaraldehyde solution.

In a sixth aspect, in addition to one or more of the preceding or following aspects, or in the alternative to some aspects, compressing at least a portion of valve leaflet tabs includes reducing a thickness of the valve leaflet tabs by at least 50% of a starting thickness.

In a seventh aspect, in addition to one or more of the preceding or following aspects, or in the alternative to some aspects, compressing at least a portion of valve leaflet tabs includes applying pressure to the valve leaflet tabs.

In an eighth aspect, in addition to one or more of the preceding or following aspects, or in the alternative to some aspects, applying pressure to the valve leaflet tabs includes applying a force to the valve leaflet tabs using a set of plates.

In a ninth aspect, in addition to one or more of the preceding or following aspects, or in the alternative to some aspects, the set of plates can include one or more surface channels.

In a tenth aspect, in addition to one or more of the preceding or following aspects, or in the alternative to some aspects, the set of plates can include one or more apertures.

In an eleventh aspect, in addition to one or more of the preceding or following aspects, or in the alternative to some aspects, applying pressure to the valve leaflet tabs includes applying at least 5 lbf force for at least 5 minutes.

In a twelfth aspect, in addition to one or more of the preceding or following aspects, or in the alternative to some aspects, applying pressure to the valve leaflet tabs includes applying at least 10 lbf force for at least 10 minutes.

In a thirteenth aspect, in addition to one or more of the preceding or following aspects, or in the alternative to some aspects, applying pressure to the valve leaflet tabs includes applying at least 20 lbf force for at least 15 minutes.

In a fourteenth aspect, in addition to one or more of the preceding or following aspects, or in the alternative to some aspects, expanding the compressed valve leaflet tabs includes allowing the valve leaflet tabs to absorb a liquid.

In a fifteenth aspect, in addition to one or more of the preceding or following aspects, or in the alternative to some aspects, the method can further include cutting away at least a portion of the expanded portions of the valve.

In a sixteenth aspect, in addition to one or more of the preceding or following aspects, or in the alternative to some aspects, the method can further include attaching the expanded valve leaflet tabs to the valve frame.

In a seventeenth aspect, in addition to one or more of the preceding or following aspects, or in the alternative to some aspects, attaching the expanded valve leaflet tabs to the valve frame includes suturing the valve leaflet tabs to the valve frame.

In an eighteenth aspect, a method of making an implantable prosthetic valve is included. The method can include compressing at least a portion of valve leaflet tabs, inserting the compressed valve leaflet tabs into commissural slots of a valve frame, and expanding the compressed valve leaflet tabs.

In a nineteenth aspect, in addition to one or more of the preceding or following aspects, or in the alternative to some aspects, compressing at least a portion of valve leaflet tabs includes removing liquid from the valve leaflet tabs.

In a twentieth aspect, in addition to one or more of the preceding or following aspects, or in the alternative to some aspects, removing the liquid from the valve leaflet tabs includes removing at least 50% by weight of the starting liquid content.

In a twenty-first aspect, in addition to one or more of the preceding or following aspects, or in the alternative to some aspects, the liquid can include an aqueous solution.

In a twenty-second aspect, in addition to one or more of the preceding or following aspects, or in the alternative to some aspects, the liquid can include a glutaraldehyde solution.

In a twenty-third aspect, in addition to one or more of the preceding or following aspects, or in the alternative to some aspects, compressing at least a portion of valve leaflet tabs includes reducing a thickness of the valve leaflet tabs by at least 50% of a starting thickness.

In a twenty-fourth aspect, in addition to one or more of the preceding or following aspects, or in the alternative to some aspects, compressing at least a portion of valve leaflet tabs includes applying pressure to the valve leaflet tabs.

In a twenty-fifth aspect, in addition to one or more of the preceding or following aspects, or in the alternative to some aspects, applying pressure to the valve leaflet tabs includes applying a force to the valve leaflet tabs using a set of plates.

In a twenty-sixth aspect, in addition to one or more of the preceding or following aspects, or in the alternative to some aspects, the set of plates include one or more surface channels.

In a twenty-seventh aspect, in addition to one or more of the preceding or following aspects, or in the alternative to some aspects, the set of plates include one or more apertures.

In a twenty-eighth aspect, in addition to one or more of the preceding or following aspects, or in the alternative to some aspects, wherein applying pressure to the valve leaflet tabs includes applying at least 5 lbf force for at least 5 minutes.

In a twenty-ninth aspect, in addition to one or more of the preceding or following aspects, or in the alternative to some aspects, wherein applying pressure to the valve leaflet tabs includes applying at least 10 lbf force for at least 10 minutes.

In a thirtieth aspect, in addition to one or more of the preceding or following aspects, or in the alternative to some aspects, wherein applying pressure to the valve leaflet tabs includes applying at least 20 lbf force for at least 15 minutes.

In a thirty-first aspect, in addition to one or more of the preceding or following aspects, or in the alternative to some aspects, expanding the compressed valve leaflet tabs includes allowing the valve leaflet tabs to absorb a liquid.

In a thirty-second aspect, in addition to one or more of the preceding or following aspects, or in the alternative to some aspects, the method can further include cutting away at least a portion of the expanded portions of the valve.

In a thirty-third aspect, in addition to one or more of the preceding or following aspects, or in the alternative to some aspects, the method can further include attaching the expanded valve leaflet tabs to the valve frame.

In a thirty-fourth aspect, in addition to one or more of the preceding or following aspects, or in the alternative to some aspects, attaching the expanded valve leaflet tabs to the valve frame includes suturing the valve leaflet tabs to the valve frame.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a perspective view of a prosthetic heart valve in accordance with various embodiments herein.

FIG. 2 is a top view of a prosthetic heart valve in accordance with various embodiments herein.

FIG. 3 is a perspective view of a valve frame in accordance with various embodiments herein.

FIG. 4 is a front view of a portion of a valve frame in accordance with various embodiments herein.

FIG. 5 is a front view of a leaflet in accordance with various embodiments herein.

FIG. 6 is a front view of a portion of a leaflet being compressed in accordance with various embodiments herein.

FIG. 7 is a front view of a leaflet in accordance with various embodiments herein.

FIG. 8 is a cross-sectional view taken along line A-A in FIG. 7 in accordance with various embodiments herein.

FIG. 9 is a cross-section view taken along line A-A in FIG. 7 in accordance with various embodiments herein.

FIG. 10 is a schematic of leaflets being attached to a frame in accordance with various embodiments herein.

FIG. 11 is a schematic of leaflets being attached to a frame in accordance with various embodiments herein.

FIG. 12 is a schematic view of leaflets being attached to a frame in accordance with various embodiments herein.

FIG. 13 is a schematic view of leaflets being attached to a frame in accordance with various embodiments herein.

FIG. 14 is a schematic view of leaflets being attached to a frame in accordance with various embodiments herein.

FIG. 15 is a schematic view of leaflets being attached to a frame in accordance with various embodiments herein.

FIG. 16 is a schematic view of leaflets being attached to a frame in accordance with various embodiments herein.

FIG. 17 is a schematic view of leaflets being attached to a frame in accordance with various embodiments herein.

FIG. 18 is a front view of a pair of leaflets in accordance with various embodiments herein.

FIG. 19 is a cross-sectional view as taken along line B-B of FIG. 18.

FIG. 20 is a cross-sectional view of leaflet tabs along with a commissure strip as shown after compression due to a compressive force being applied thereto.

FIG. 21 is a schematic view of leaflets being attached to a frame in accordance with various embodiments herein.

FIG. 22 is a front view of a plate in accordance with various embodiments herein.

FIG. 23 is an end view of a plate in accordance with various embodiments herein.

FIG. 24 is a front view of a plate in accordance with various embodiments herein.

FIG. 25 is an end view of a plate in accordance with various embodiments herein.

FIG. 26 is a front view of a plate in accordance with various embodiments herein.

FIG. 27 is a flowchart depicting a method of attaching leaflets to a frame in accordance with various embodiments herein.

FIG. 28 is a flowchart depicting a method of attaching leaflets to a frame in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, prosthetic heart valves can be implanted to address heart valve issues and restore proper heart function. Prosthetic heart valves frequently include two or more leaflets (commonly three) that are attached to a frame. Prosthetic valves can be configured to allow one-way flow through the valve, such as by separating the leaflets from each other to open the valve thereby allowing flow and joining together (valve leaflet coaptation) to close the valve thereby blocking flow. In some cases, prosthetic valves can further include an inner skirt and/or an outer skirt to restrict the path of flow through the valve.

The assembly of a prosthetic valve can impact its function. For example, if the valve leaflets are too loose, a pinwheel effect could result in leakage between free edges of adjacent leaflets. If the valve leaflets are too tight, then inadequate sealing between adjacent leaflets could occur.

Embodiments of attaching leaflets to a frame are disclosed herein that offer benefits for consistent and proper leaflet attachment to frames. In some embodiments, the leaflets can include two mounting tabs that are separated from each other by the free edge of the leaflet. The tabs can be inserted through a portion of the frame, such as a commissural slot. However, in accordance with embodiments herein, the tabs can be compressed prior to being inserted through the frame. The compressed portion of the tab can have a reduced thickness compared to an uncompressed tab, which in some cases can help facilitate inserting the tab through a portion of the frame. The compressed portion can be inserted through commissural slot and the leaflet can be secured to the frame. Insertion of a compressed portion is easier than insertion of a non-compressed portion and makes it easier to assure that a desired amount of insertion has occurred to prevent the valve leaflet from being too loose or too tight.

In some embodiments, the compressed portion of the tab can be removed, such as by cutting, from the remainder of the leaflet tab after it has been threaded through the commissural slot. In some embodiments, the compressed portion of the tab can be expanded after the tab has been threaded through the commissural slot resulting in an interference fit attaching the leaflet to the frame. The methods provided herein can provide a more consistent arrangement of the valve leaflets with a valve frame.

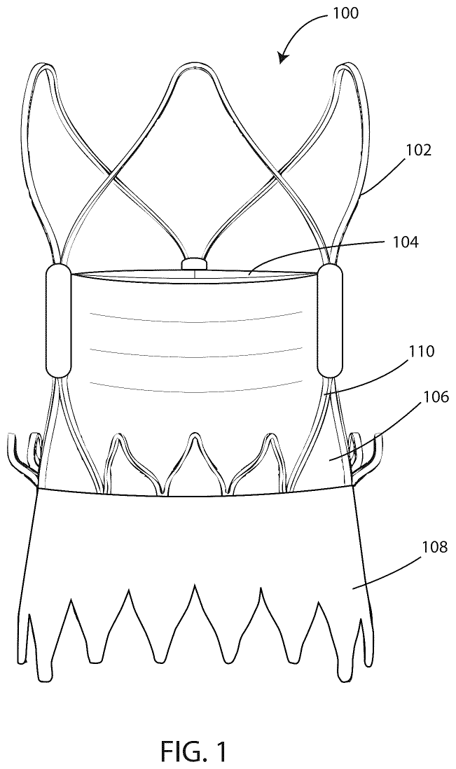

FIG. 1 is a perspective view of a prosthetic heart valve 100, according to various embodiments. The valve 100 can include a frame 102, a plurality of leaflets 104, an inner skirt 106, and an outer skirt 108. In some cases, the frame 102 can include a plurality of frame struts 110. However, frames lacking distinct struts are also contemplated herein.

In various embodiments, the implantable valve 100 can include an inner skirt 106. The inner skirt 106 can define a substantially tubular shape. The inner skirt 106 can be disposed on a luminal surface of the frame 102. The luminal surface of the frame 102 can be a surface of the frame 102 that defines a central lumen. The inner skirt 106 can direct fluid, such as blood, flowing through the valve 100. The inner skirt 106 can ensure the fluid flows through the central lumen of the valve 100 and does not flow around the leaflets 104 when the leaflets 104 are in a closed configuration.

In various embodiments, the implantable valve 100 can include an outer skirt 108. The outer skirt 108 can define a substantially tubular shape. The outer skirt 108 can be disposed on an abluminal surface of the frame 102. The abluminal surface of the frame 102 can be a surface of the frame 102 that is external to the central lumen. The outer skirt 108 can be disposed between the frame 102 and the vessel wall in order to prevent fluid, such as blood, flowing around the valve 100. The outer skirt 108 can ensure the fluid flows through the valve 100 and does not flow around the valve 100, such as to ensure that the leaflets 104 can stop the flow of fluid when in a closed position.

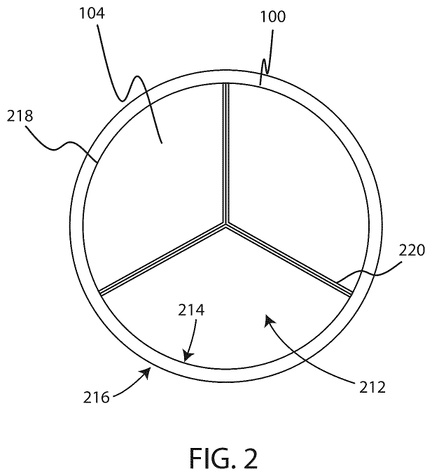

FIG. 2 shows an end view of the closed valve 100 according to various embodiments. It should be appreciated that the valve 100 can be any type of heart valve (e.g., a mitral valve, an aortic valve, etc.). In use, the valve 100 can be implanted (e.g., surgically or through transcatheter delivery) in a mammalian heart. The valve 100 can be configured to allow one-way flow through the valve 100.

The valve 100 can include a frame 102 defining a central lumen 212 which, in some embodiments, can be substantially cylindrical, but can also take on other shapes. The side of the frame 102 and other components facing the central lumen 212 can be referred to as the luminal surface 214 or luminal side. The opposite side of the frame 102 and other components (e.g., facing away from the central lumen 212) can be referred to as the abluminal surface 216 or abluminal side. In various embodiments, the frame 102 can have a substantially circular cross-section. In other embodiments, the frame 102 can have a non-circular, such as a D-shaped, cross-section. In some embodiments, a non-circular frame 102 can be used to repair a mitral valve or another non-circular valve in the body. The frame 102, including at least some components thereof, can be formed of various materials including, but not limited to, metals and metal alloys, such as corrosion resistant metals and metal alloys, composites, ceramics, polymers, and the like. In some embodiments, the frame 102 can be at least partially formed of nitinol or NiTiCo.

The valve 100 can include a plurality of leaflets 104 disposed within the central lumen 212. Each leaflet 104 can include a respective root edge 218 coupled to the frame 102 and a respective free edge or coaptation edge 220 movable relative to the root edge 218 to coapt with the coaptation edges 220 of the other leaflets 104 along the coaptation region.

The coaptation edges 220 of the leaflets 104 move into coaptation with one another in a closed position (FIG. 2) to substantially restrict fluid from flowing past the valve 100. Specifically, the leaflets 104 can coapt to fill up or close the central lumen of the valve 100 thereby impeding the flow of fluid.

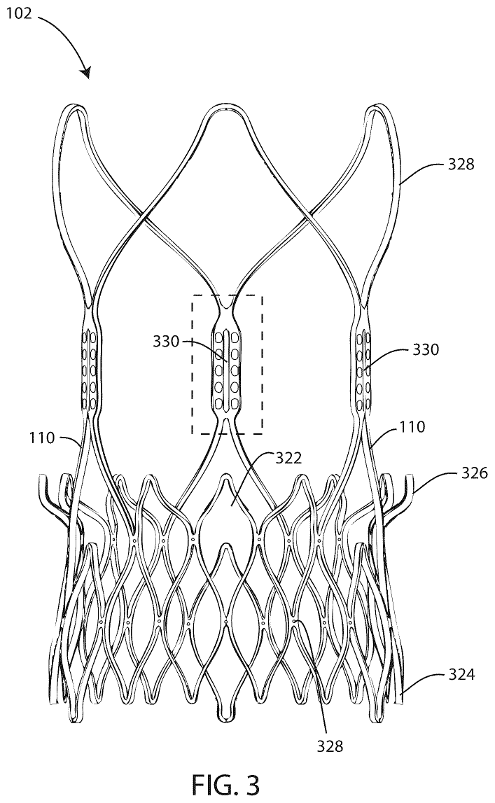

FIG. 3 shows a schematic view of a frame 102, according to various embodiments. The frame can include the plurality of frame struts 110. The frame struts 110 can be rods, bars, plates, or other projections that define a framework or lattice structure. In some embodiments, the frame can define open spaces 322 between the frame struts 110. However, in other embodiments the frame may not include such open spaces 322.

In some embodiments, the frame struts 110 can define a lower crown 324, an upper crown 326 and a plurality of stabilization arches 328. In some embodiments, the inner skirt 106 can be coupled to the lower crown 324 and/or the upper crown 326. In other embodiments, the inner skirt 106 is only coupled to the upper crown 326.

In some embodiments, the outer skirt 108 can be coupled to the lower crown 324 and/or the upper crown 326. In other embodiments, the outer skirt 108 is only coupled to the lower crown 324. In some embodiments, the valve leaflets 104 can be coupled to the frame 102 at a position that is at or just below the stabilization arches 328 and above the upper crown 326.

In various embodiments, at least one of the frame struts 110 can define a connection aperture 330, such as a commissural slot. A connection aperture 330 can be configured to receive a connection portion or a projection (such as a mounting tab) from a valve leaflet 104 to couple the leaflet 104 to the frame 102. In various embodiments, the frame 102 can define at least three connection apertures 330.

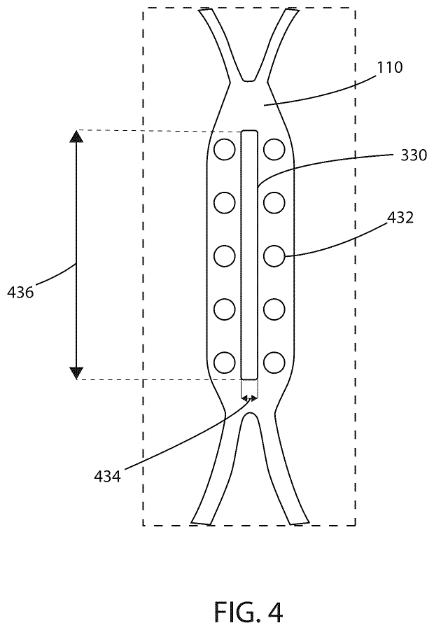

FIG. 4 shows a front view of a connection aperture 330 in accordance with various embodiments herein. In some embodiments, the connection aperture 330 can be substantially rectangular, but can take on various shapes.

In some embodiments, the connection aperture 330 can have a width 434 and a length 436. The width 434 and the length 436 can be sized to accept two mounting tabs, one mounting tabs from each of two adjacent leaflets 104. In some embodiments, the width 434 can be at least twice the size of the thickness of a single leaflet tab, such as to be able to accommodate two leaflet tabs. In some embodiments, the length 436 can be approximately equal to the height of a leaflet tab, such as to allow a leaflet tab to be inserted into the connection aperture 330.

In some embodiments, the frame strut 110 can further define suture apertures 432. The suture apertures 432 can be defined on either side of the connection aperture 330. In some embodiments, as will be described later, a leaflet tab can be inserted through the connection aperture and sutured into place with the frame 102. The suture apertures 432 can accommodate the sutures coupling the leaflet tab to the frame 102.

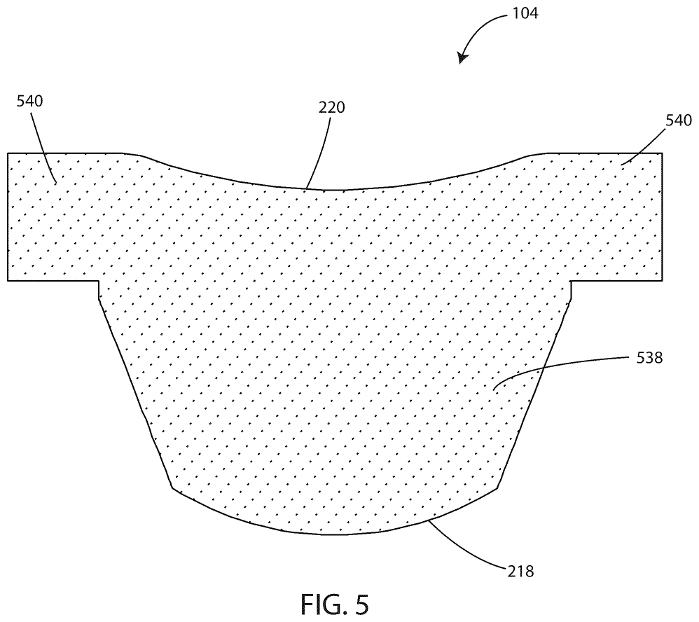

FIG. 5 shows a front view of a leaflet 104 in accordance with various embodiments herein. The leaflet 104 shown in FIG. 5 can represent an uncompressed leaflet that has a substantially universal thickness. The valve leaflets 104 can include a coaptation edge or free edge 220. The valve leaflets 104 can further include two connection portions or tabs 540. One tab 540 can be disposed on either end of the free edge 220. The free edge 220 can extend from one tab 540 to the other tab 540. In some cases, the connections portions may also be referred to as commissural mounting tabs.

In some embodiments, the tabs 540 can eventually be disposed at least partially within a connection aperture 330 defined by a portion of the frame 102 thereby coupling or attaching the leaflet 104 to the frame 102. In some embodiments, tabs 540 can be projections extending away from a leaflet body 538. The tabs 540 can be integral with the other portions of the valve leaflet 104, such that the leaflet 104 is a single part or monolithic element.

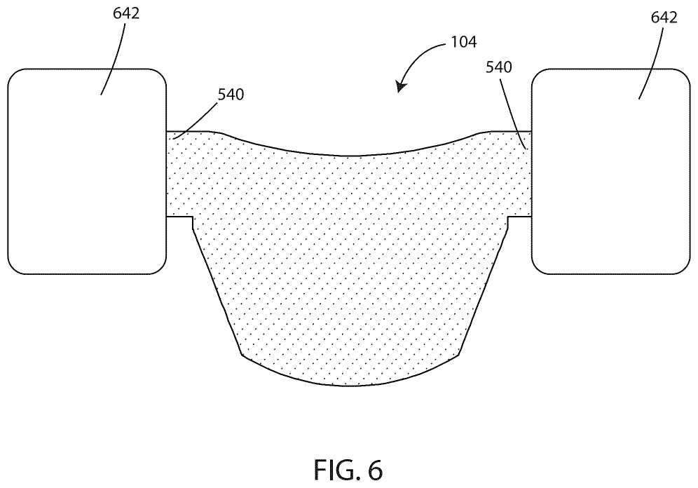

Prior to coupling a valve leaflet 104 with a valve frame 102, the valve leaflet can be prepared for coupling. In some embodiments, a portion of the valve leaflet 104, such as a portion of the tabs 540, can be compressed. FIG. 6 shows a front view of a portion of a leaflet 104 being compressed in accordance with various embodiments herein. Compressing a tab 540, or a portion of a leaflet 104, can include applying pressure to the tab 540 or portion of the leaflet 104.

Tabs can be compressed in various ways using plates, clamps, or the like. For example, in some embodiments a tab 540 of the leaflet 104 can be compressed between two plates 642. In various embodiments, the plates 642 can be substantially flat. In various embodiments, the plates 642 can be identical or mirrored versions of each other. In various embodiments, the plates 642 can include an upper plate and a lower plate. In some embodiments, one upper plate and one lower plate can compress both tabs 540 simultaneously. In some embodiments, one upper plate and one lower plate can compress one tab 540, and a second upper plate and a second lower plate can compress the other tab 540. FIG. 6 shows two plates 642 compressing two different portions of the leaflet 104. Each of the two plates shown can have an additional plate disposed below it, which is not visible in FIG. 6.

An uncompressed tab 540 can be disposed between an upper plate and a lower plate. The plates can move toward each other. In some embodiments, one of the two plates is stationary, while the second plate is translated or moved towards the stationary plate. In some embodiments, both plates can translate or move towards a common central area to compress the portion of the tab that is disposed between the two plates.

FIG. 6 shows an example a first set of plates 642 compressing a portion of one tab 540 and second set of plates 642 compressing a portion of the other tab 540. In some embodiments, the first tab 540 can be compressed simultaneously with the second tab. In some embodiments, one of the tabs 540 is compressed and then the other tab is compressed. In some embodiments, the sets of plates can be attached to one another (not shown) to facilitate simultaneously compressing both tabs. In some embodiments, a plate 642 can be in the form of a stainless steel block. In various embodiments, the plate can have a substantially flat inner surface, such as the surface that contacts the tab 540 and/or the commissure strip. In various embodiments, the plate 642 can include an inner surface that defines one or more surface features, such as those described below with regards to FIGS. 20-25.

In some embodiments, the force provided for compressing the leaflet 104 can be at least 5 lbf force, at least 10 lbf force, or at least 15 lbf force. In some embodiments, the leaflet 104 can be compressed for at least 5 minutes, at least 10 minutes, at least 15 minutes or at least 20 minutes.

In some embodiments, compressing the leaflet 104 can include applying 5 lbf force for at least 5 minutes. In some embodiments, compressing the leaflet 104 can include applying at least 10 lbf force for at least 10 minutes. In some embodiments, compressing the leaflet 104 can include applying at least 20 lbf force for at least 15 minutes.

In some embodiments, compressing the leaflet 104 can include applying a 7N load for about 10 minutes, such as to achieve about 40 psi pressure on the tissue. In some embodiments, a load of about 7N, 14N, or 21N can be applied. In some embodiments, a load of at least 5N, of at least 10N, of at least 15N, of at least 20 N, or of at least 25N can be applied.

In various embodiments, when the portion of the leaflet 104 is compressed, liquid can be removed from the leaflet 104. In some embodiments, liquid from the compressed portions can be removed from the compressed portions. In some embodiments, compressing the leaflet tabs 540 can include removing at least 20, 30, 40, 50, 60, or 70% or more of the starting liquid content from the tab 540, or an amount falling within a range between any of the foregoing. In some embodiments, the liquid can include an aqueous solution. In some embodiments, the liquid can include a glutaraldehyde solution.

After the leaflet 104 has been compressed, the plates 642 can be withdrawn or separated to release the leaflet 104. The leaflet can include compressed portions and uncompressed portions.

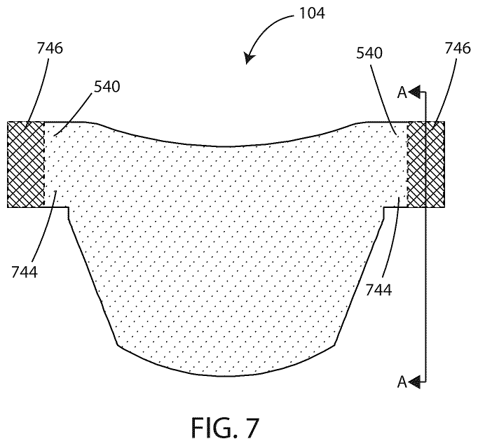

FIG. 7 shows a front view of a leaflet 104 in accordance with various embodiments herein. The leaflet 104 can include tabs 540. In some embodiments, the tabs 540 can include an uncompressed portion 744 and a compressed portion 746. In various embodiments, the compressed portion 746 can be distal to the uncompressed portion 744.

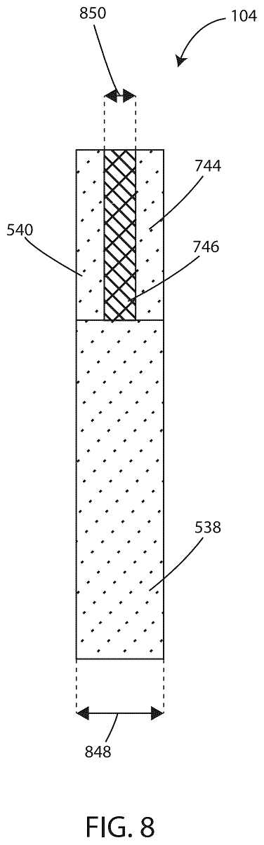

The compressed portion(s) of the leaflet 104 can have a reduced thickness, such as shown in FIG. 8. FIG. 8 shows a cross-sectional view taken along line A-A in FIG. 7 in accordance with various embodiments herein.

The uncompressed portion 744 and the leaflet body 538 can have a similar thickness 848. The compressed portion 746 can have a thickness 850 that is less than the uncompressed portion 744 and the leaflet body 538.

In some embodiments, the compressed portion 746 can have a thickness 850 that is at least 10, 20, 30, 40, 50, 60, 70, or 80% smaller than the uncompressed portion 744, or an amount falling within a range between any of the foregoing, such that the starting thickness of the compressed portion 746 has been reduced by at least 50%. In some embodiments, the compressed portion 746 can have a thickness 850 that is at least 10% smaller than the uncompressed portion 744. In some embodiments, the compressed portion 746 can have a thickness 850 that is at least 25% smaller than the uncompressed portion 744. In some embodiments, the compressed portion 746 can have a thickness 850 that is at least 75% smaller than the uncompressed portion 744.

It will be appreciated that compression herein can be symmetric or asymmetric. For example, the compression shown in FIG. 8 is symmetric in that when viewed in cross-section the width of the compressed portion 746 lines up with a central portion of the width of the uncompressed portion 744 (such as may result from the leaflet being compressed by two opposed plates applying force onto the leaflet tabs from opposite sides of the thickness thereof). However, in other embodiments, compression can be asymmetric such that the compressed portion is disposed to one side of the leaflet with respect to a thickness thereof (such as may result from the leaflet being supported by a large flat surface and a smaller plate pushing down on a portion of the leaflet from the opposite side).

In some embodiments, multiple tabs 540 can be compressed at the same time in preparation for assembling the tabs 540 with a frame 102. In various embodiments, two adjacent tabs 540 can be surrounded by a commissure strip 910 prior to compressing the tabs 540. The commissure strip 910 can be applied or wrapped around the tabs 540 of two adjacent leaflets 104. The commissure strip 910 can cap or surround the end of the two tabs 540.

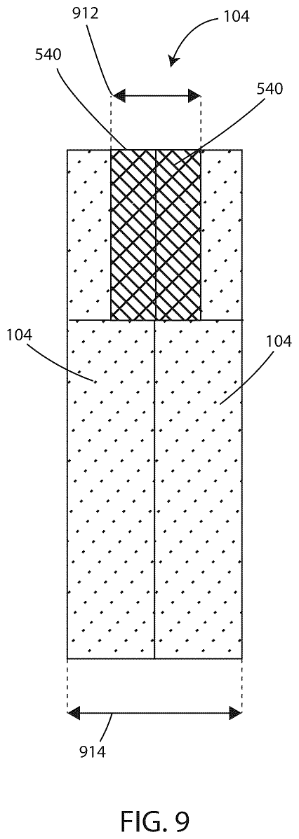

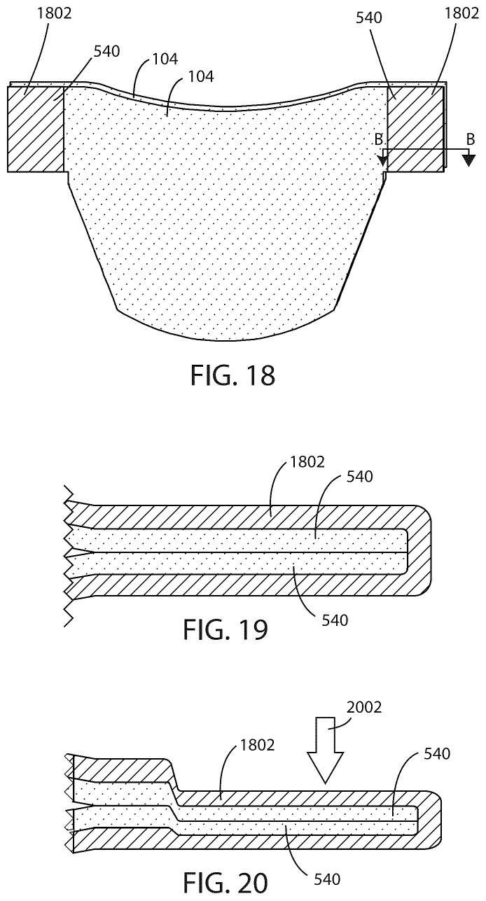

In various embodiments, the two tabs 540 and the commissure strip 910 can be compressed, such as between a set of plates 642 as shown in FIG. 6. FIG. 9 shows a cross-sectional view taken along line A-A in FIG. 7 according to some embodiments. FIG. 9 shows two adjacent leaflets 104. The two tabs (and, in some embodiments a commissure strip described below with reference to FIGS. 18-20) have been compressed resulting in a thickness 912. The thickness 912 of the two tabs 540 can be less than the width 434 of the connection aperture 330, such that the tabs 540 can be inserted into the connection aperture 33.

In various embodiments, the thickness of an uncompressed tabs 540 can be represented by thickness 914. In some embodiments, the uncompressed thickness 914 can be thicker than the compressed thickness of the leaflets 104 by twice the compressed thickness. In various embodiments, the compressed thickness 912 can be less than 75% of the uncompressed thickness 914. In various embodiments, the compressed thickness 912 can be less than 70% of the uncompressed thickness 914. In various embodiments, the compressed thickness 912 can be less than 60% of the uncompressed thickness 914. In various embodiments, the compressed thickness 912 can be less than 50% of the uncompressed thickness 914. In various embodiments, the compressed thickness 912 can be less than 40% of the uncompressed thickness 914. In various embodiments, the compressed thickness 912 can be less than 30% of the uncompressed thickness 914. In various embodiments, the compressed thickness 912 can be less than 20% of the uncompressed thickness 914.

In some embodiments, the uncompressed thickness 914 of the tabs 540 (and in some embodiments a commissure strip) can be about 800 μm. In some embodiments, the compressed thickness 912 can be between about 350 μm to 480 μm.

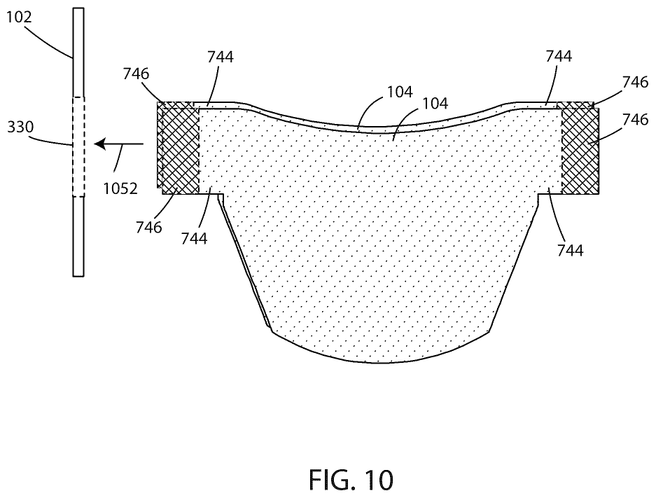

The leaflet 104 including compressed portions 746 can be coupled aligned with a valve frame 102. The compressed portions 746 of the tabs 540 can be aligned with the connection aperture 330 to facilitate coupling the leaflet 104 with the frame 102 as shown in FIG. 10. FIG. 10 shows a schematic of leaflets 104 being aligned with a connection aperture 330 of a frame 102 in accordance with various embodiments herein.

In various embodiments, two leaflets 104 can be aligned with one connection aperture 330. The tabs 540 of the leaflets 104 can be at least partially inserted into and through the connection aperture 330. The leaflets 104 can move in the direction of arrow 1052 to insert the tabs 540 into the connection aperture 330. It should be understood that while two leaflets are shown being aligned with the frame at the same time in FIG. 10, it is possible that one leaflet is aligned and inserted into the connection aperture at a time.

### Attaching Leaflets to Frame

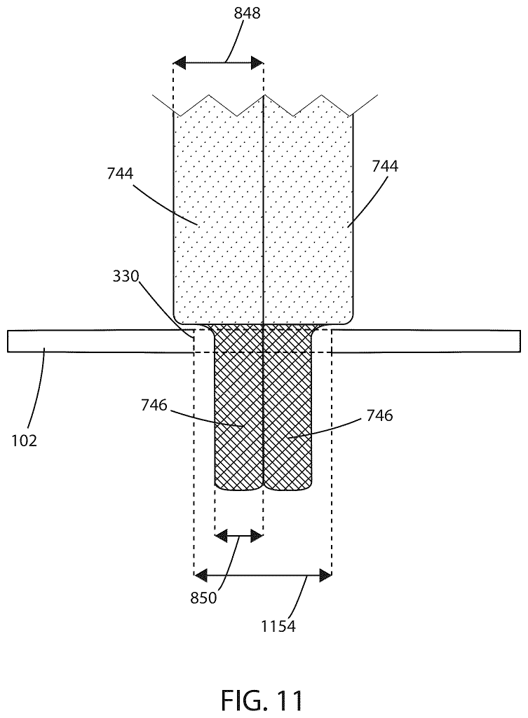

FIG. 11 shows a schematic cross-section view of the leaflets 104 inserted into the connection aperture 330 in accordance with various embodiments herein. In various embodiments, the width 1154 of the connection aperture 330 can be about 600 μm, however, in some embodiments it could be wider or narrower. In some embodiments, the nominal width of a pair of leaflet tabs (and a commissure strip where included as described below) before compression can be about 800 μm. However, in some embodiments the nominal width of a pair of leaflet tabs (and a commissure strip where included as described below) before compression can be wider or narrower, such as 600, 625, 650, 675, 700, 725, 750, 775, 800, 850, or 900 μm, or more or less, or a width falling within a range between any of the foregoing.

As shown in FIG. 11, in some embodiments, the width 1154 of the connection aperture 330 can be at least twice the thickness 850 of the compressed portions 746, such as to accommodate the tabs 540 from two adjacent leaflets 104. The compressed portion 746 of the leaflet 104 can have a thickness 850 that is less than half of the width 1154 of the connection aperture 33. In some embodiments, the width 1154 of the connection aperture can be less than the thickness 848 of two uncompressed portions 744. In some embodiments, inserting a tab 540 into the connection aperture 330 can include only inserting the compressed portion 746 into the connection aperture 330. In some embodiments, inserting a tab 540 into the connection aperture 330 can include inserting the compressed portion 746 and at least a portion of the uncompressed 744 tab 540 into the connection aperture. In various embodiments, both of the tabs 540 are inserted into the connection aperture 330 the same amount, such that the distal ends of the tabs 540 align with each other through the connection aperture 330.

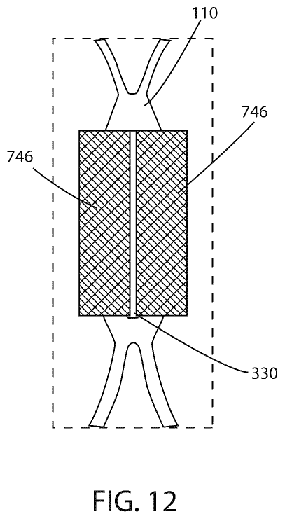

FIG. 12 shows a schematic view of two compressed portions 746 of leaflets 104 that have been threaded through the connection aperture 330 in accordance with various embodiments herein. In various embodiments, after the compressed portions 746 of the leaflets have been threaded through the connection aperture 330, a portion of the uncompressed portion 744 can be threaded through the connection aperture 330.

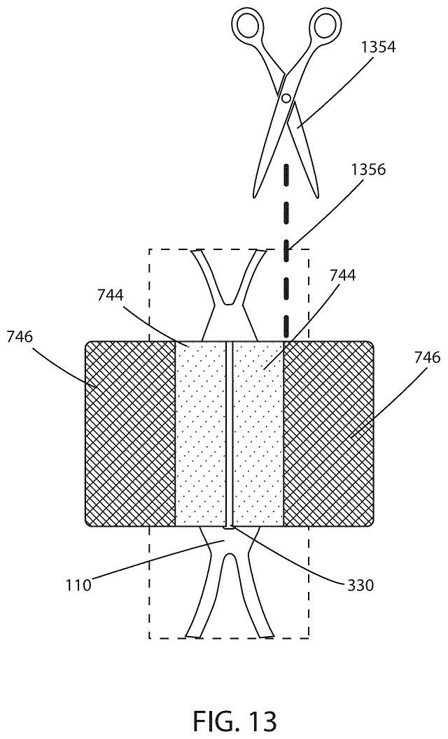

FIG. 13 shows a schematic view of portions of two uncompressed portions 744 that have been threaded through the connection aperture 330 in accordance with various embodiments herein.

In various embodiments, the compressed portions 746 can be separated from the uncompressed portions 744. For example, in some embodiments, a portion of the compressed portion 746 can be trimmed or cut away. In some embodiments, the entirety of the compressed portion 746 can be trimmed, cut away, or otherwise separated.

In various embodiments, the compressed portion 746 can be separated, such as by cutting, from the uncompressed portion 744. In some embodiments, a scissors 1354, a blade, or other cutting instrument can be used to separate the compressed portion 746 from the uncompressed portion 744, such as along line 1356 shown in FIG. 13.

While FIG. 13 only shows one of the two leaflets being cut, it should be understood that both leaflets can be cut in a similar way. In some embodiments, one or multiple cutting instruments 1354 can be used simultaneously to separate the compressed portions 746 from the uncompressed portions 744.

After the compressed portions 746 have been separated from the uncompressed portions 744, the compressed portions 746 can be discarded. As a result, in some embodiments, the uncompressed portions 744 can remain threaded through the connection aperture, such as shown in FIG. 14.

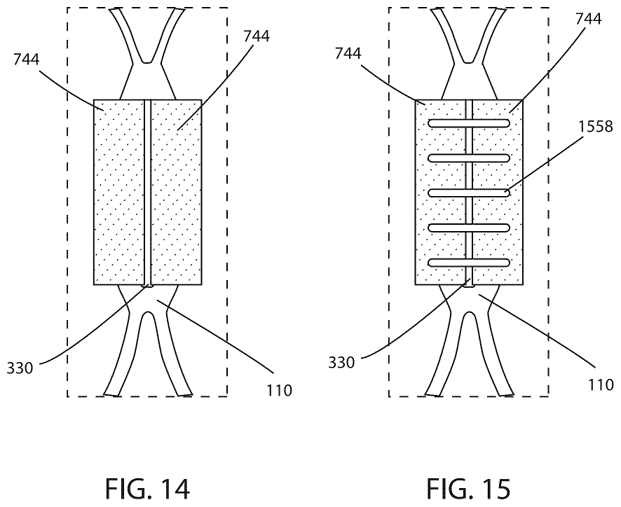

The remaining portions of the tabs 540, such as the uncompressed portions 744, can be secured to the frame 102, such as with sutures 1558 (or other attachment mechanisms) as shown in FIG. 15. In various embodiments, the leaflets 104 can be sutured to the frame 102, such as with sutures 1558 extending through suture apertures 432. In various embodiments, suturing the leaflets to the frame can include suturing a leaflet to an adjacent leaflet as shown in FIG. 15. In some embodiments, suture the leaflets to the frame can include suturing the tab 540 to itself to encircle a portion of the frame 102.

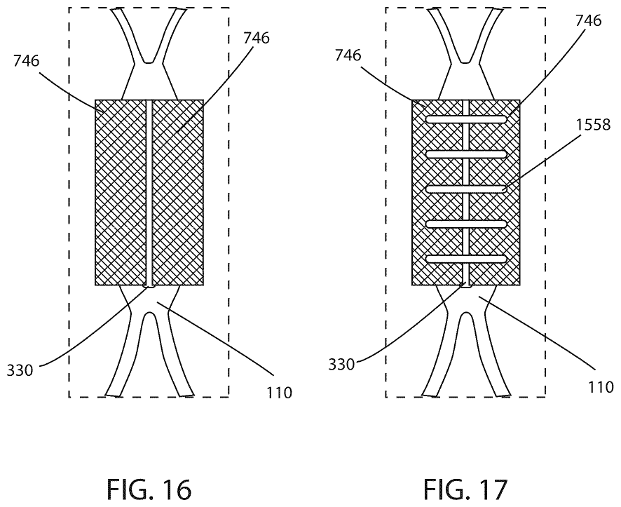

In alternative embodiments, the compressed portion 746 can remain attached to the tab 540. FIG. 16 shows a schematic view of the compressed portions 746 of two leaflets being attached to a frame in accordance with various embodiments herein. In some embodiments, only the compressed portion 746 extends through the connection aperture 330. In other embodiments, the compressed portion 746 as well as at least some of the uncompressed portion 744 extends through the connection aperture 330.

The portions of the tabs 540 that extend through the connection aperture, such as the compressed portions 746, can be secured to the frame 102. In some embodiments, the portions of the tabs 540 that extend through the connection aperture 330 can be secured to the frame with sutures 1558 as shown in FIG. 17. In various embodiments, the leaflets 104 can be sutured to the frame 102, such as with sutures 1558 extending through suture apertures 432. In various embodiments, suturing the leaflets to the frame can include suturing a leaflet to an adjacent leaflet as shown in FIG. 17. In some embodiments, suturing the leaflets 104 to the frame 102 can include suturing the tab 540, such as the compressed portion 746, to itself to encircle a portion of the frame 102. It will be appreciated that leaflets can also be attached to frames using mechanisms/approaches beyond sutures such as adhesives, clips, clamps, pins, and the like or combinations thereof.

### Commissure Strips

In various embodiments herein, commissure strips can be disposed over leaflet tabs. Commissure strips can be used to prevent leaflet tabs from contacting the internal sides of the connection aperture of the frame. Commissure strips can be formed of various materials, such as a rectangular piece of porcine that wraps around the leaflet tabs. As such, in some embodiments both a commissure strip and the leaflet tabs are pulled through the frame post slot.

Referring now to FIG. 18, a front view of a pair of leaflets 104 is shown in accordance with various embodiments herein. The leaflets 104 can include tabs 540. Commissure strips 1802 can be disposed over at least a portion of the tabs 540.

Referring now to FIG. 19, a cross-sectional view is shown as taken along line B-B of FIG. 18. In this view, a commissure strip 1802 is disposed over the tabs 540 and wraps around an end thereof. The commissure strip 1802 can be formed of various materials including natural materials, synthetic materials, and semi-synthetic materials. In various embodiments, the commissure strip 1802 can be derived from a natural tissue. In various embodiments, the commissure strip 1802 can be a compressible material.

In some cases, leaflet tabs can be compressed separately from a commissure strip. However, in many cases, when a commissure strip is used, the commissure strip can be positioned over the leaflet tabs and then the combined leaflet tabs and commissure strip can be compressed. Referring now to FIG. 20, a cross-sectional view of leaflet tabs 540 along with a commissure strip 540 is shown after compression due to a compressive force 2002 being applied thereto.

### Expanding Compressed Portions

In some embodiments, compressed portions can be expanded back (e.g., decompressed) at various points in the valve assembly process. FIG. 11, as described above, shows a schematic cross-section view of the leaflets 104 inserted into the connection aperture 330 in accordance with various embodiments herein. As shown in FIG. 11, the compressed portions 746 can extend through the connection aperture 330. In some embodiments, the leaflets 104 can be attached to the frame 102 with an interference fit.

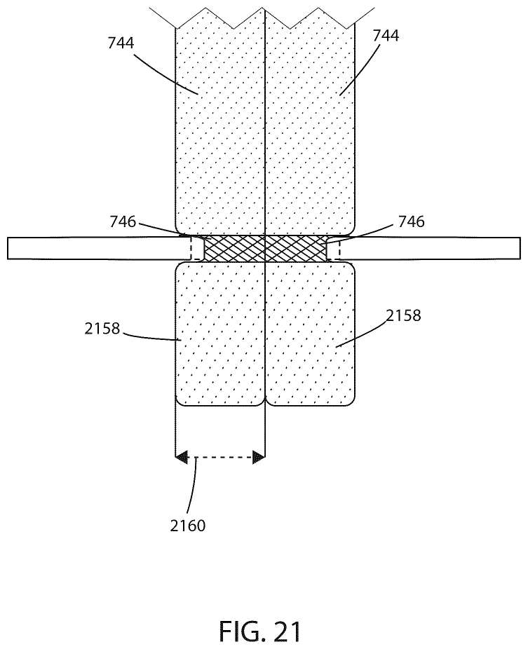

In some embodiments, to attach the leaflet 104 to the frame 102, at least a portion of the compressed portion 746 can be expanded as shown in FIG. 21. FIG. 21 shows a schematic view of leaflets 104 being attached to a frame 102 with an interference fit in accordance with various embodiments herein.

In some embodiments, the compressed portion of the valve leaflet tab can be expended. In some embodiments, expanding the compressed portion includes allowing the leaflet to absorb liquid. In some embodiments, only a portion of the compressed portion is expanded, such that the valve leaflet 104 can include an uncompressed portion 744, a compressed portion 746, and an expanded portion 2158. In other embodiments, the entire compressed portion 746 can be expanded resulting in the valve leaflet 104 including an uncompressed portion 744 and an expanded portion 2158.

After expanding the compressed portions, the expanded portions 2158 can have a thickness 2160 that is larger than the compressed state and, in some embodiments, approximately equal to the uncompressed portions 744 or the thickness of the compressed portion prior to being compressed. The expanded portions 2158 can have a thickness 2160 that is approximately equal to the pre-compressed thickness or at least 95, 90, 80, or 70 percent thereof.

In some embodiments, a portion of the expanded portion 2158 can be cut away or otherwise separated from the remainder of the leaflet 104. In some embodiments, the expanded portions 2158 can be attached to the valve frame 102. In some embodiments, the expanded portions 2158 can be attached to the valve frame 102 with a plurality of sutures.

In various embodiments, the interference fit can be a result of the expanded portions 2158 having a thickness 2160 that is greater than half of the width 1154 of the connection aperture 330. An expanded portion 2158 can prevent an adjacent expanded portion from withdrawing through the connection aperture 330, such as by physically interfering with the expanded portions movement through the connection aperture 330.

### Compression Plates

FIGS. 20-24 show various embodiments of the inner surface of a plate 642. As mentioned above, one or more leaflets and a commissure strip can be compressed between plates 642. In some embodiments, the inner surface of a plate 642, such as the surface that contacts the leaflet and/or commissure strip, can be substantially flat.

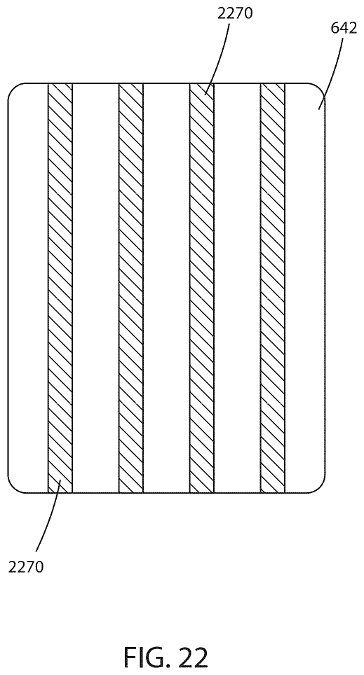

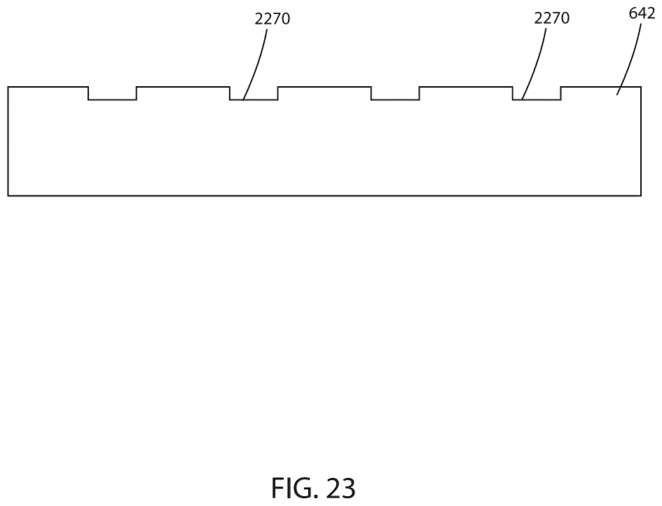

In some embodiments, a plate 642 can define one or more channels 2270. FIG. 22 shows a front view of an inner surface of a plate 642 in accordance with various embodiments. FIG. 23 shows an end view of the plate 642 shown in FIG. 22. In some embodiments, the channels 2270 can all be aligned in the same direction, such as shown in FIG. 22.

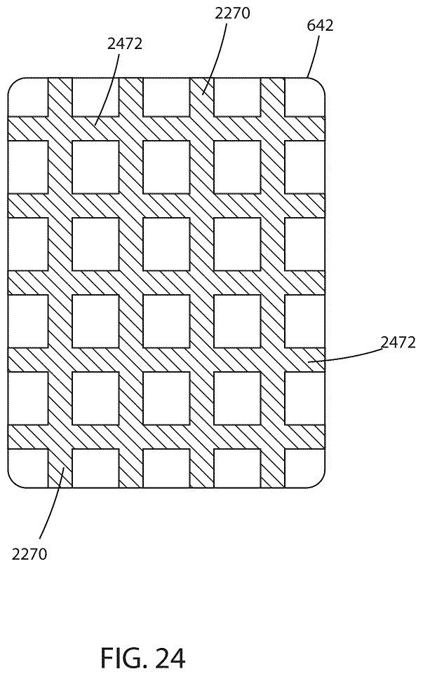

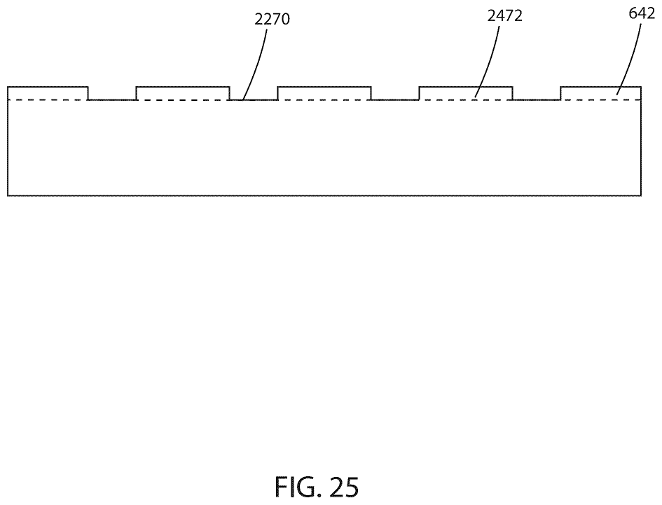

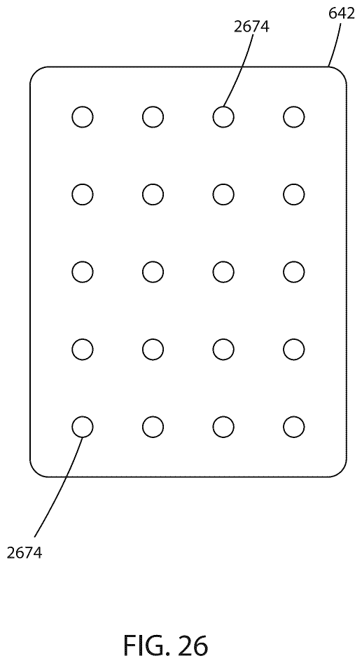

In some embodiments, channels can be aligned in multiple directions, such as shown in FIG. 24. FIG. 24 shows a front view of an inner surface of a plate 642 in accordance with various embodiments. FIG. 25 shows an end view of the plate 642 shown in FIG. 24. The plate 642 can define one or more vertical channels 2270 and one or more horizontal channels 2472. In various embodiments, the channels 2270, 2472 can facilitate the removal of moisture from the tissue while it is being compressed. As the tissue is compressed, fluid can be expelled from the tissue. The fluid can be removed from the compression area through the channels 2270, 2472. In various embodiments, the channels 2270, 2472 can cover less than 25%, 20% 15%, 5%, 3%, 2%, or 1% of the surface area of the inner surface of the plate 642.

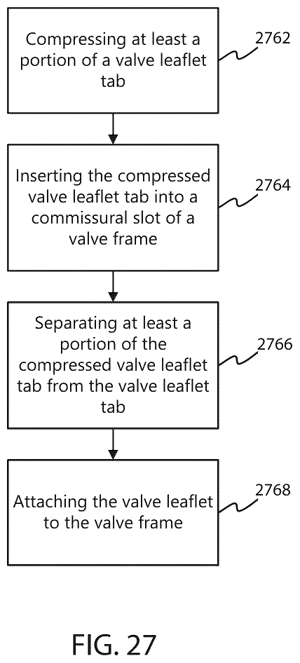

FIG. 27 shows a front view of an inner surface of a plate 642 in accordance with various embodiments. In various embodiments, a plate 642 can define one or more hydration apertures 2674. The hydration apertures 2674 can be configured to ensure that the leaflet tissue is hydrated with glutaraldehyde through the compression process. In some embodiments, a supply of glutaraldehyde can be in fluid communication with the hydration apertures 2674. Glutaraldehyde can be pumped from the supply to the plate 642, such as to provide glutaraldehyde to the leaflet tissue during compression.

In various embodiments, the hydration apertures 2674 can each have a diameter of about 0.5 mm. In various embodiments, the hydration apertures 2674 can be spaced apart by approximately 3 mm. In various embodiments, a plate 642 can include the hydration apertures 2674 shown in FIG. 27 and channels 2270, 2472 shown in FIGS. 20- 24.

### Methods

Various methods for attaching a valve leaflet to a valve frame are provided herein. FIG. 27 shows a flowchart depicting a method of attaching leaflets to a frame in accordance with various embodiments herein.

The method for making an implantable prosthetic valve can include compressing at least a portion of a valve leaflet tab of a valve leaflet 2762. In various embodiments, compressing the valve leaflet tab can include applying a pressure to the valve leaflet tab for a predetermined time period. In various embodiments, compressing the valve leaflet tab can include removing liquid from the leaflet tab. In various embodiments, compressing the leaflet can reduce the thickness of the leaflet by at least 50% compared to a starting thickness of the leaflet.

After the valve leaflet tab has been compressed, the method can include inserting the compressed valve leaflet tab into a commissural slot of a valve frame 2764. In some embodiments, inserting the compressed valve leaflet tab into the commissural slot can include inserting two compressed valve leaflet tabs into the same commissural slot.

After the valve leaflet tab has been inserted into the commissural slot, the method can include separating at least a portion of the compressed valve leaflet tab from the valve leaflet tab 2766. In some embodiments, the compressed valve leaflet tab can be separated from the uncompressed valve leaflet tab by cutting. In some embodiments, separating the compressed portion of the valve leaflet tab can include separating the entire compressed portion from the uncompressed portion of the valve leaflet tab.

After the compressed portion has been separated from the uncompressed portion valve leaflet tab, the method can include attaching the valve leaflet to the valve frame 2768. In some embodiments, attaching the leaflet to the frame can include suturing the remaining portion of the valve leaflet tab to itself, to the frame, and/or to an adjacent leaflet tab.

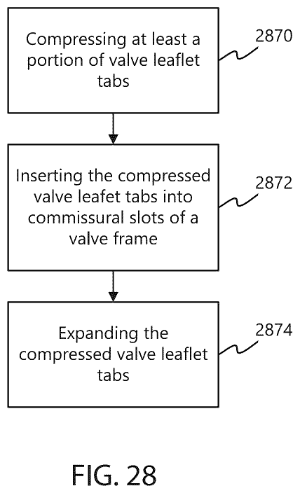

FIG. 28 shows a flowchart depicting a method of attaching leaflets to a frame in accordance with various embodiments herein.

The method for making an implantable prosthetic valve can include compressing at least a portion of a valve leaflet tab of a valve leaflet 2870. In various embodiments, compressing the valve leaflet tab can include applying a pressure to the valve leaflet tab for a predetermined time period. In various embodiments, compressing the valve leaflet tab can include removing liquid from the leaflet tab. In various embodiments, compressing the leaflet can reduce the thickness of the leaflet by at least 50% compared to a starting thickness of the leaflet.

After the valve leaflet tab has been compressed, the method can include inserting the compressed valve leaflet tab into a commissural slot of a valve frame 2872. In some embodiments, inserting the compressed valve leaflet tab into the commissural slot can include inserting two compressed valve leaflet tabs into the same commissural slot.

After the valve leaflet tab has been inserted into the commissural slot, the method can include expanding the compressed valve leaflet tabs 2874. In various embodiments, expanding the compressed valve leaflet tabs includes allowing the valve leaflet tabs to absorb a liquid. In various embodiments, the method can further include attaching the expanded valve leaflet tabs to the valve frame with an interference fit and/or a sutures.

### Leaflet Materials

In various embodiments, the leaflets, and the skirt (the inner skirt and/or the outer skirt) can include an animal tissue. In various embodiments, the animal tissue can include at least one of porcine tissue and bovine tissue. In some embodiments, the animal tissue may include pericardial tissue. In various embodiments, the animal tissue can include at least about 0.15% by weight of a cation of a salt. In various embodiments, the animal tissue can include at least about 0.3% by weight of a cation of a salt. In various embodiments, the cation can include at least one of sodium, potassium, calcium and magnesium.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method of making an implantable prosthetic valve comprising:
compressing at least a portion of valve leaflet tabs;
inserting the compressed valve leaflet tabs into commissural slots of a valve frame; and
expanding the compressed valve leaflet tabs.

2. The method of making an implantable prosthetic valve of claim 1, wherein compressing at least a portion of valve leaflet tabs comprises removing liquid from the valve leaflet tabs.

3. The method of making an implantable prosthetic valve of claim 2, wherein removing the liquid from the valve leaflet tabs comprises removing at least 50% of the starting liquid content.

4. The method of making an implantable prosthetic valve of claim 2, the liquid comprising an aqueous solution.

5. The method of making an implantable prosthetic valve of claim 2, the liquid comprising a glutaraldehyde solution.

6. The method of making an implantable prosthetic valve of claim 1, wherein compressing at least a portion of valve leaflet tabs comprises reducing a thickness of the valve leaflet tabs by at least 50% of a starting thickness.

7. The method of making an implantable prosthetic valve of claim 1, wherein compressing at least a portion of valve leaflet tabs comprises applying pressure to the valve leaflet tabs.

8. The method of making an implantable prosthetic valve of claim 7, wherein applying pressure to the valve leaflet tabs comprises applying a force to the valve leaflet tabs using a set of plates.

9. The method of making an implantable prosthetic valve of claim 8, wherein the set of plates comprise one or more surface channels and/or one or more apertures.

10. The method of making an implantable prosthetic valve of claim 7, wherein applying pressure to the valve leaflet tabs comprises applying at least 5 lbf force for at least 5 minutes.

11. The method of making an implantable prosthetic valve of claim 1, further comprising cutting away at least a portion of the expanded portions of the valve.

12. The method of making an implantable prosthetic valve of claim 1, further comprising attaching the expanded valve leaflet tabs to the valve frame.

13. A method of making an implantable prosthetic valve comprising:
compressing at least a portion of valve leaflet tabs;
inserting the compressed valve leaflet tabs into commissural slots of a valve frame; and
expanding the compressed valve leaflet tabs.

14. The method of making an implantable prosthetic valve of claim 13, wherein compressing at least a portion of valve leaflet tabs comprises removing liquid from the valve leaflet tabs.

15. The method of making an implantable prosthetic valve of claim 14, wherein removing the liquid from the valve leaflet tabs comprises removing at least 50% of the starting liquid content.

16. The method of making an implantable prosthetic valve of claim 15, the liquid comprising a glutaraldehyde solution.

17. The method of making an implantable prosthetic valve of claim 13, wherein compressing at least a portion of valve leaflet tabs comprises reducing a thickness of the valve leaflet tabs by at least 50% of a starting thickness.

18. The method of making an implantable prosthetic valve of claim 13, wherein compressing at least a portion of valve leaflet tabs comprises applying at least 5 lbf force for at least 5 minutes.

19. The method of making an implantable prosthetic valve of claim 13, further comprising cutting away at least a portion of the expanded portions of the valve.

20. The method of making an implantable prosthetic valve of claim 13, further comprising attaching the expanded valve leaflet tabs to the valve frame.
